(19) United States
(12) Patent Application Publication
(10) Pub. No.: US 2023/0157646 A1
(43) Pub. Date: May 25, 2023

(54) CONTACTLESS MONITORING OF PHOTOPLETHYSMOGRAPHY USING RADAR

(71) Applicant: Koko Home, Inc., Palo Alto, CA (US)

(72) Inventors: Luca Rigazio, Los Gatos, CA (US); Usman Mohammed Khan, Raleigh, NC (US); Sheen Kao, East Palo Alto, CA (US)

(21) Appl. No.: 17/992,031

(22) Filed: Nov. 22, 2022

Related U.S. Application Data
(60) Provisional application No. 63/282,332, filed on Nov. 23, 2021.

(51) Int. Cl. A61B 5/00; A61B 5/05

(52) U.S. Cl. CPC A61B 5/7278; A61B 5/05; A61B 5/7267; A61B 5/7203

(57) ABSTRACT

A contactless method for monitoring photoplethysmography in a human comprises illuminating the human with radiofrequency energy from a transmitter without contacting the patient with the transmitter, sensing the radiofrequency energy reflected back from the human with at least one antenna, and using an artificial neural network to generate a photoplethysmography waveform based on the reflected energy.

### CROSS-REFERENCE TO RELATED APPLICATIONS

This application claims priority to and incorporates by reference U.S. patent application Ser. No. 63/282,332 filed Nov. 23, 2021.

### TECHNICAL FIELD

This disclosure relates to photoplethysmography (PPG) and more particularly to monitoring PPG with radar.

### BACKGROUND

Regular monitoring of human physiological systems such as breathing and cardiac activity is important both in-hospital and at-home due to its importance in medical diagnosis as well as patient monitoring. One of the gold standards of such monitoring is PPG.

PPG is an optical technique that detects changes in blood volume through a pulse oximeter that illuminates the skin and measures changes in light absorption. The ability to monitor PPG easily and at scale for a large population allows for better pre-screening of many health conditions, and also improves the overall general well-being of the individuals. It has been broadly used for monitoring hypertension, measuring cardiac output, predicting cardiovascular disease risk, and for early screening of different pathologies. Moreover, different features derived from PPG are used as diagnostics for conditions such as arterial stiffness, estimated risk of coronary heart disease, presence of atherosclerotic disorders, etc.

### BRIEF SUMMARY

In one aspect, a contactless method for monitoring photoplethysmography in a human comprises illuminating the human with radiofrequency energy from a transmitter without contacting the patient with the transmitter, sensing the radiofrequency energy reflected back from the human with at least one antenna, and using an artificial neural network to generate a photoplethysmography waveform based on the reflected energy.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a block diagram illustrating a PPG system, in accordance with some examples.

FIG. 2 is a diagrammatic representation of a processing environment, in accordance with some examples.

FIG. 3 is a block diagram illustrating an artificial neural network, in accordance with some examples.

FIG. 4 illustrates charts showing the effects of bandpass filtering, in accordance with some examples.

FIG. 5 is a block diagram illustrating the encoder-decoder model of the artificial neural network, in accordance with some examples.

FIG. 6 illustrates multipath scenarios, in accordance with some examples.

FIG. 7 is a block diagram illustrating a self-attention model, in accordance with some examples.

FIG. 8 illustrates a method for monitoring photoplethysmography in a human, according to some examples.

FIG. 9 is a block diagram showing a software architecture within which the present disclosure may be implemented, according to some examples.

FIG. 10 is a diagrammatic representation of a machine in the form of a computer system within which a set of instructions may be executed for causing the machine to perform any one or more of the methodologies discussed herein, in accordance with some examples.

### DETAILED DESCRIPTION

Examples disclosed herein provide a radio frequency based contactless approach that accurately estimates a PPG signal (interchangeably also referred to as PPG waveform) using radar for stationary participants. Changes in the blood volume that manifest in the PPG waveform are correlated to the physical movements of the heart, which the radar can capture. To estimate the PPG waveform, examples use a self-attention architecture to identify the most informative reflections in an unsupervised manner, and then uses an encoder decoder network to transform the radar phase profile to the PPG sequence.

One of the use-cases of PPG is in monitoring the cardiac cycle, which involves pumping of blood from heart to the body. PPG captures the variations in blood volume in the skin during the diastolic and systolic phases of the cardiac cycle. In the diastolic phase, the heart muscles relax, the chambers of the heart fill with blood, and the blood pressure decreases. In contrast, the heart muscles contract in the systolic phase, the blood gets pushed out to different organs, and the blood pressure increases. Therefore, the changes in the blood volume that manifest in the PPG waveform are correlated to the physical movements of the heart, which the radar captures.

As the signal to noise ration (SNR) of the signal reflected from the heart is extremely small, the radar signal at an antenna may only show the systolic and diastolic movements at a few points in time only. For example, the systolic movement may only be visible for a small part of one cardiac cycle, and may only be visible a few cycles later. Similarly, as there can be multiple antennas, some movements may be more visible at certain antennas compared to others at any given time. A deep learning network such as a Convolutional Neural Network (CNN) is used to exploit this property by using spatial filters to extract different patterns that are correlated to systolic and diastolic movements.

In addition, deep learning models leverage the diversity of multipath reflections as each multipath will have a distinct angle with the heart movements. A deep learning model also uses both extrapolation and interpolation. If the prediction window of the deep learning model is long enough such that it contains multiple cardiac cycles, then the model can learn to extrapolate information from one cardiac cycle to another. Similarly, the model can learn to interpolate by filling in the missing movement patterns for any given cardiac cycle.

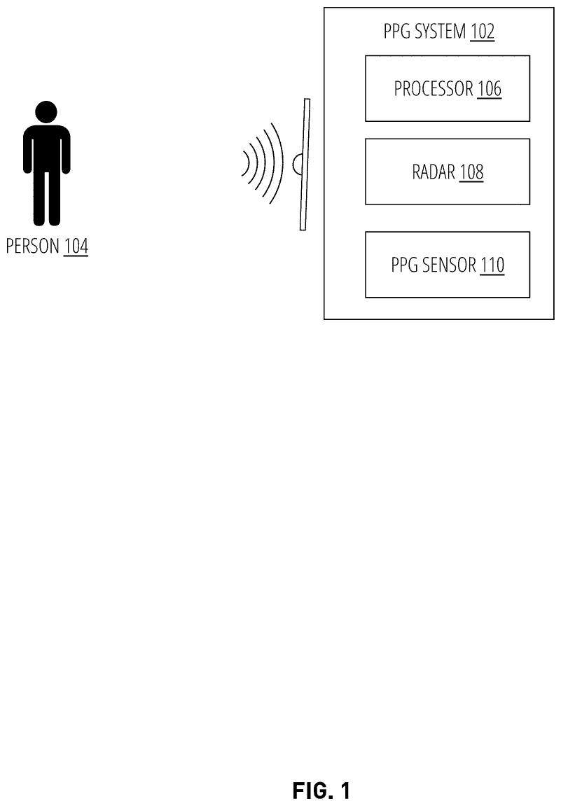

FIG. 1 is a block diagram illustrating a PPG system 102, in accordance with some examples. The PPG system 102 comprises a processor 106 hosting an artificial neural network 300(e.g., deep learning based encoder decoder model), a radar 108, which comprises one or more each of a transmit antenna and a receive antenna, and optionally a PPG sensor 110. The PPG system 102, in one example, the radar 108 includes a Frequency Modulated Carrier Wave (FMCW) radar, which transmits radio frequency signals, and receives reflections of the transmitted radio frequency signals from a person 104. If the person 104 or persons are stationary, then the primary changes in the radar signal are caused by the small breathing and heartbeat movements. The optional PPG sensor 110 can be used during a training phase as will be discussed further below. The PPG sensor 110 can be wearable and comprises a light source and a photodetector worn at the surface of skin to measure volumetric variations of blood circulation. The PPG system 102 uses a deep-learning based encoder decoder model that transforms these small movements contained in the radar signal to the PPG waveform, as will be discussed further below.

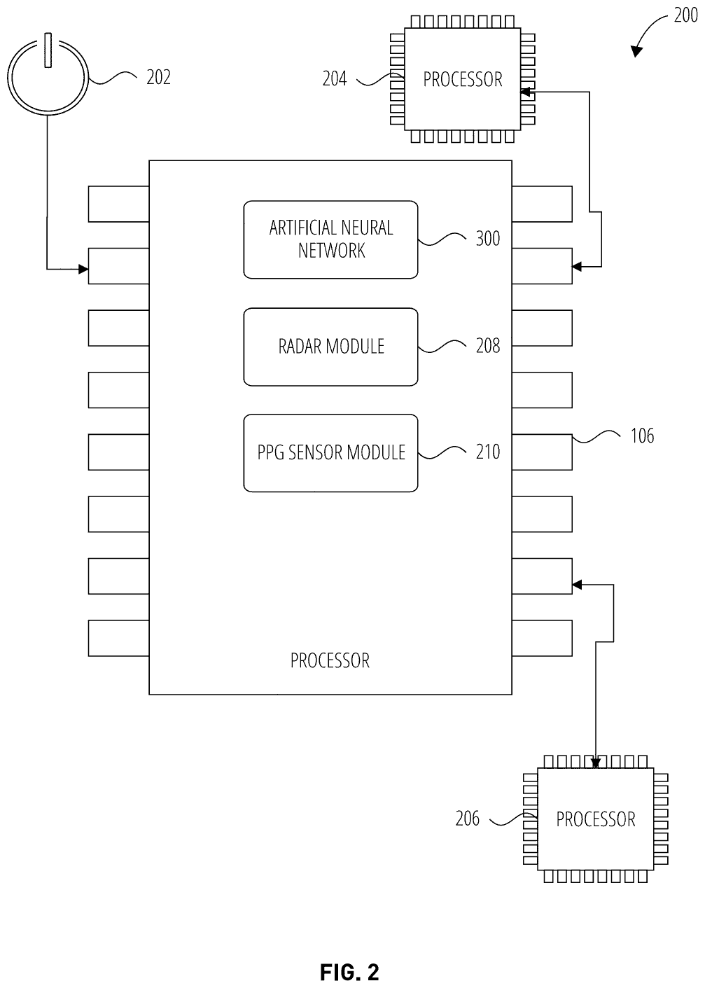

Turning now to FIG. 2, a diagrammatic representation of a processing environment 200 is shown, which includes the processor 204, the processor 206, and a processor 106 (e.g., a GPU, CPU, etc., or combination thereof).

The Processor 106 is shown to be coupled to a power source 202, and to include (either permanently configured or temporarily instantiated) modules, namely an artificial neural network 300, a radar module 208, and a PPG sensor module 210. The artificial neural network 300 operationally generates a PPG waveform based on data received from the radar 108; the radar module 208 operationally generates, using the radar 108, radiofrequency signals and receives reflected signals for analysis by the artificial neural network 300, and the PPG sensor module 210 operationally generates, using the PPG sensor 110, PPG data for training the artificial neural network 300. As illustrated, the processor 106 is communicatively coupled to both the processor 204 and processor 206, and the modules can be distributed between the processors.

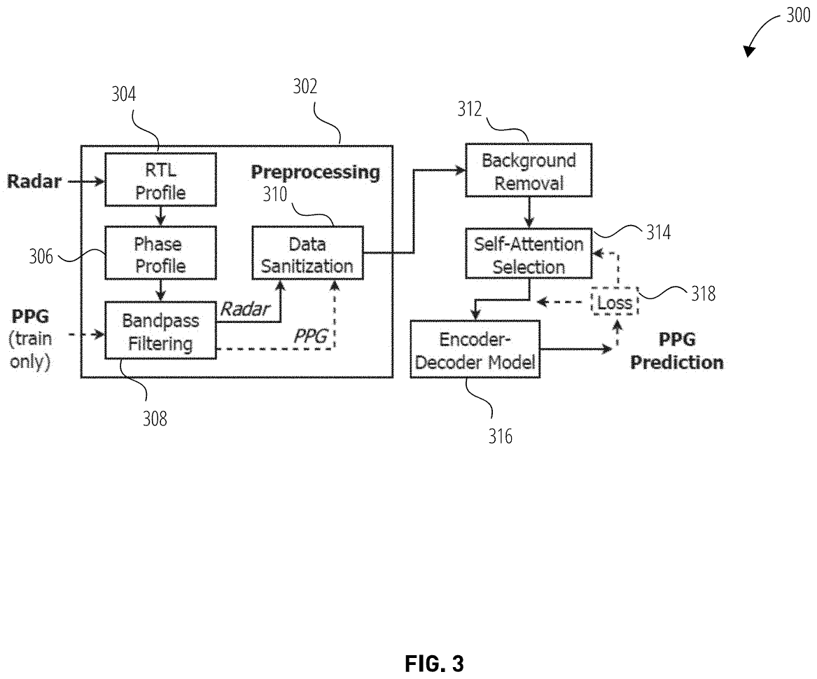

FIG. 3 is a block diagram illustrating the artificial neural network 300, in accordance with some examples. The artificial neural network 300 comprises preprocessing 302, background removal 312, self-attention selection 314 and encoder-decoder model 316. In the preprocessing 302, the artificial neural network 300 receives a stream of continuous data from both the radar 108 and the PPG sensors 110. The preprocessing 302 prepares small synchronized chunks of these streams such that they can be fed to the encoder-decoder model 316. To prepare radar data, the artificial neural network 300 estimates the Round Trip Length RTL profile 304 that indicates the RTL of each reflection that is received at the radar 108. Next, the artificial neural network 300 estimates the phase of RTL profiles over a time window, and obtains the phase profile 306. As the phase of the radar signal is affected by small chest and heart movements, the phase profile 306 can capture these movements. The artificial neural network 300 then applies bandpass filtering 308 on both the radar phase profiles 306 and the ground truth PPG signal from the PPG sensor 110 to obtain breathing and heartbeat signals for both modalities. The motivation for applying the bandpass filtering 308 is to ensure that the signals from the two modalities look as similar as possible, as well as to remove any high frequency noise to help learning. The final preprocessing step is to apply data sanity checks, e.g., data sanitization 310, to ensure that the encoder-decoder model 316 does not train on erroneous data instances such as when the person 104 is moving, or is not correctly wearing the ground truth PPG measurement PPG sensor 110.

The background removal 312 differentiates the primary participant (person 104) from any background participants, and discards background reflections if present. To discard the reflections from background participants, the artificial neural network 300 first identifies all RTL bins from stationary participants using a periodicity heuristic. The artificial neural network 300 then marks the closest RTL bin from a stationary participant as the representative bin, and measures the similarity of all other stationary RTL bins with the representative bin using Dynamic Time Warping (DTW). Finally, the artificial neural network 300 filters the background RTL bins by setting them to zero in the input radar representation.

The self-attention selection 314 downsizes the number of RTL bins, as many of these bins do not contain any useful signal, rather only represent noise. To obtain a downsized representation, the artificial neural network 300 computes an attention map, and then projects it to the radar input to obtain a representation that only contains the most informative RTL bins. An attention map can be a scalar matrix that represents the relative importance of each RTL bin with respect to the target task of predicting the output PPG signal. Instead of using heuristics to select the appropriate RTL bins, the artificial neural network 300 uses a self-attention based learning architecture that integrates within the overall model, and learns to translate the input radar representation to the downsized representation of selective RTL bins.

The encoder-decoder model 316 transforms the downsized radar phase profile sequence obtained from the previous step to the output PPG time series sequence. The artificial neural network 300 uses a convolutional encoder decoder architecture, where both the encoder and decoder are implemented using CNNs. The convolutional encoder captures progressively high level features as the receptive fields of the network increase with the depth of the encoder. In contrast, the decoder progressively increases the spatial resolution of the feature maps through up-sampling.

There are at least three technical challenges facing the artificial neural network 300. The first challenge involves designing a good loss function for the learning network. A straightforward loss that computes the element-wise distance between the ground-truth and the predicted PPG sequences does not work well for two reasons. First, small synchronization errors between PPG sensor 110 data and radar 108 data, which prevent the model from converging. To address this challenge, the artificial neural network 300 uses a synchronization invariant loss function that slides the target PPG sequence by different offsets, computes the [symbol] 1 loss on each slide, and then selects the smallest loss value while discarding the rest. Second, the PPG signal is flip-invariant, while the radar signal is not. This is because as a participant turns around to face the opposite direction from the radar, the radar phase signal also flips around. However, at the same time, the position of the person does not impact the PPG signal in any way. To address this challenge, the artificial neural network 300 modifies the loss function such that it carries this flip-invariance property.

The second challenge is that a majority of the RTL bins in the radar phase profile 306 do not contain any reflections from the person 104, rather only represent noise. Therefore, training the encoder-decoder model 316 with all the RTL bins will not only unnecessarily increase its complexity, but will also make it prone to overfitting. To address this challenge, the self-attention selection 314 learns to identify the most informative RTL bins with respect to the target task of predicting the output PPG signal. Moreover, the self-attention selection 314 itself learns, and integrates within the encoder-decoder model 316 to avoid adding any unnecessary complexity.

The third challenge is that there may be multiple participants in the environment beside the primary participant that PPG system 102 is tracking. To address this challenge, the artificial neural network 300 identifies all RTL bins from the stationary participants, and then uses Dynamic Time Warping (DTW) technique to measure the similarity of different RTL bins with a representative bin that is closest to the PPG system 102. Subsequently, the artificial neural network 300 filters the background RTL bins by setting them to zero in the input radar representation. However, another challenge that arises with this approach is that it is difficult to generate a large dataset with background participants. To address this challenge, the artificial neural network 300 uses an augmentation strategy where it randomly sets a few RTL bins to zero. Thus, the artificial neural network 300 can simulate the multi-person scenario even when a single person is present in the environment.

The radar 108 transmits a sinusoidal chirp and receives reflections from the environment. The frequency of the transmitted chirp increases linearly with time at a rate m. On the receiving side, a mixer multiplies the signal received at time t with the signal transmitted at time t to downconvert it. If the received signal is comprised of L direct and multipath reflections, where the RTL of the i^{th} reflection is dᵢ, then the received signal at time t, after passing through the mixer, is given as

$$y ( t ) = {\sum }_{i = 1}^{L} cos [ 2 {π f}_{i} t + ( {ϕ}_{T} - {ϕ}_{i} ) ] , where {f}_{i} = m \frac{{d}_{i}}{c} , {ϕ}_{T}$$

is the initial phase of the transmitted signal, and Øᵢ is the received phase of the i^{th} reflection. This expression shows that each reflection that travels an RTL of dᵢ meters introduces a frequency

$${f}_{i} = m \frac{{d}_{i}}{c}$$

in the downconverted signal y(t). Thus, the magnitude of FFT of y(t) will have L peaks, where each peak represents the frequency introduced by one of the L reflections into y(t). The complex valued FFT of y(t) is represented as ŷ(f), which may be the RTL profile of the environment because each frequency fᵢ in this FFT represents an RTL equal to

$$c \frac{{f}_{i}}{m} ,$$

any value of this RTL profile at any given frequency fₖ, i.e., ŷ(f=fₖ) denotes an RTL bin, which quantifies the magnitude and phase of the signal with frequency fₖ arriving at the radar. If there are N antennas, then we can get an RTL profile ŷ^{j}(f) for each antenna 9, where 1≤j≤N.

After the artificial neural network 300 estimates the RTL profiles, it proceeds by extracting the phases of each RTL profile bin ŷ(f=fₖ) over a time window W. The phases capture the small chest and heart movements that a person makes even when they are stationary. In particular, the phase of an RTL profile bin for a given antenna at a time instance t is represented as Ø^{j}(t,f), and is given by

$$2 π \frac{d ( t )}{λ} .$$

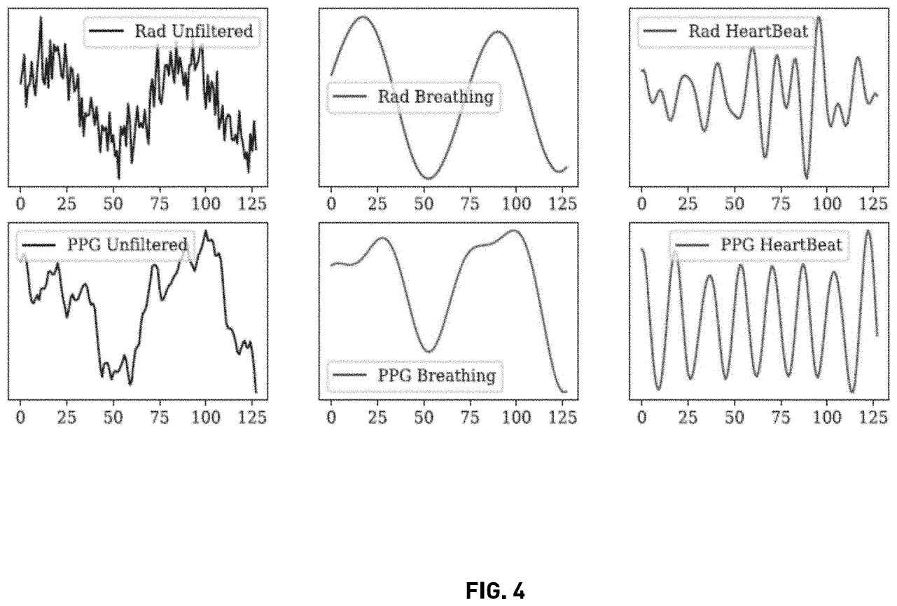

In this expression, λ denotes the wavelength of the transmitted signal, and d(t) is the round trip distance between the person 104 and the PPG system 102. As d(t) changes during exhales, inhales, as well as during different cycles of the heartbeat, Ø^{j}(t,f) captures these movements. An example sliced phase profile for some fixed values of j and f is shown in FIG. 4.

The preprocessing 302 makes the two representations—PPG sensor 110 data and radar 108 phase profile data—as similar as possible. For example, if the two signals have different sampling rates, the artificial neural network 300 will use a more complex model that first learns to re-sample the two signals. Therefore, to avoid making the model unnecessarily complex, the artificial neural network 300 re-sample both signals at a common sampling frequency fₛ, which is set to 20 Hz in one example. Further, while the breathing harmonic is dominant in a radar phase profile, the heartbeat harmonic dominates the breathing harmonic in the PPG signal. This can be seen in the unfiltered radar and PPG signals shown in FIG. 4.

In FIG. 4, the top row (left to right) shows: Ø^{j}(t,f) for fixed values of j and f, breathing radar phase profile Ø_{b}^{j}(t,f), and heartbeat radar phase profile Øₕ^{j}(t,f). The bottom row (left to right) shows: PPG signal p(t), breathing PPG profile p_{b}(t), and heartbeat PPG profile pₕ(t).

Therefore, a learning model may not be very effective if it is trained directly on the unfiltered radar phase profiles. Instead, for each window, the artificial neural network 300 obtains two bandpass filtered signals each for both the radar phase profile and the PPG signal. Using the bandpass filtering 308 obtains similar breathing and heartbeat signals for radar and PPG, which the encoder-decoder model 316 can then learn to translate. Let Ø_{b}^{j}(t,f) and Øₕ^{j}(t,f) denote the breathing and heartbeat radar phase profiles, respectively. To obtain these profiles, the artificial neural network 300 used Butterworth band-pass filters with cutoff frequencies of [0.2, 0.6]Hz and [0.8, 3.5]Hz, respectively. The Butterworth filter provides a maximally flat response in the pass-band. Similarly, let the PPG signal be denoted as p(t), then the breathing and heartbeat PPG signals are represented as p_{b}(t) and pₕ(t), respectively. The combined breathing and heartbeat signals for both radar and PPG is denoted as Ø_{b|h}^{j}(t,f) and p_{b|h}(t), respectively. FIG. 4 shows these signals after bandpass filtering for both radar and PPG. Therefore, an objective of the encoder-decoder model 316 is to obtain the following transformation:

ϕ_{b|h}^{j}(t,f)→p_{b|h}(t)

Finally, another advantage of these bandpass filters is that they remove any sensor or environment specific high frequency noises, which may otherwise adversely affect the encoder-decoder model 316 performance by causing it to overfit.

Returning to FIG. 3, The final preprocessing 302 step of the artificial neural network 300 is data sanitization 310 to check for data sanity to ensure that the encoder-decoder model 316 does not train on erroneous data. There are three sanity checks that the artificial neural network 300 makes in this step. First, the artificial neural network 300 ensures that the person 104 who is generating data for training the model is actually wearing the PPG sensor 110. To carry out this check, the artificial neural network 300 discards a data sample if the dynamic range of the PPG signal p(t) is below a certain threshold, since it indicates that the PPG signal does not change over time. Second, the artificial neural network 300 ensures that the person is stationary by discarding any data samples where the dynamic range of the PPG signal is above a certain threshold. As these thresholds are sensor-specific, their values can be calibrated through experiments with the specific PPG sensor used in the implementation. The third and final sanity check is to ensure that the person is within the range and field of view of the radar 108. To carry out this check, the artificial neural network 300 uses a periodicity heuristic that determines if the dominant motion in the radar signal is due to breathing.

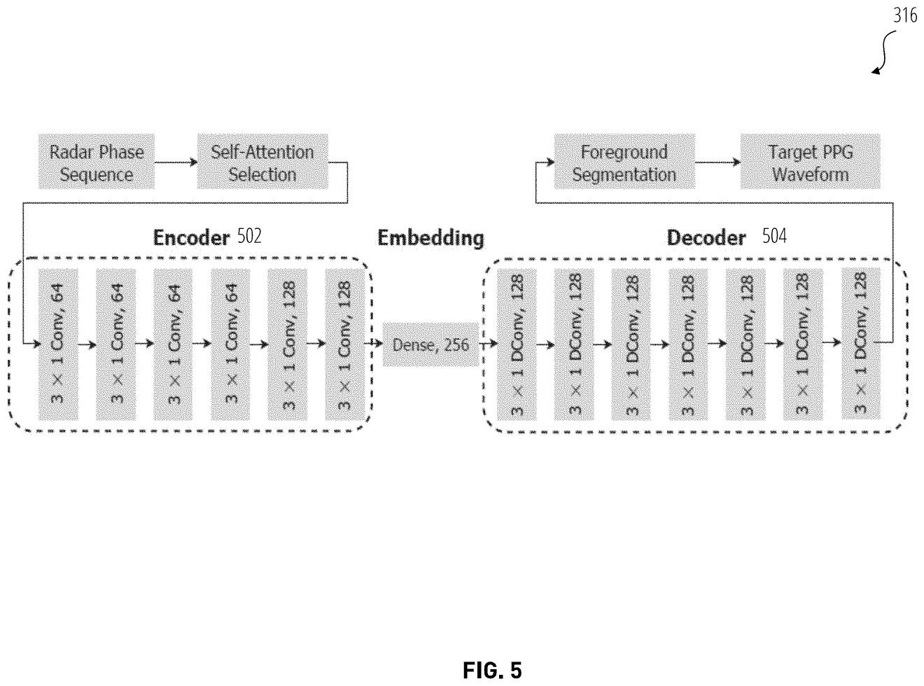

FIG. 5 is a block diagram illustrating the encoder-decoder model 316 of the artificial neural network 300, in accordance with some examples. The artificial neural network 300 takes the phase profile sequence Ø_{b|h}^{j}(t,f) as input, and predicts the output PPG sequence p_{b|h}(t). Recall that the shape of Ø_{b|h}^{j}(t,f) is (W, N, F, 2), where F is the number of RTL bins, and the last dimension indicates the breathing and heartbeat bandpass filtered signals. Similarly, the shape of p_{b|h}(t) is (W, 2). In one example, F is set to 64, which means that the last RTL bin denotes a distance of roughly 2.5 m. However, a majority of these RTL bins do not contain any reflections from the person 104, rather they only represent noise. Therefore, training the encoder-decoder model 316 with all the RTL bins will not only unnecessarily increase its complexity, but will also make it prone to overfitting. An approach to address this issue is to use a heuristic that identifies the location of the person 104, and then selects the corresponding single RTL bin. However, as shown below, this will make the encoder-decoder model 316 lose a lot of information that it can potentially exploit. Moreover, a heuristic-based selection of a single RTL bin tends to be error-prone, and does not generalize well to different environments. Therefore, the artificial neural network 300 trains the self-attention selection 314 model that learns to identify the top RTL bins that contain the most useful information, and then feed only those RTL bins to the encoder-decoder model 316, as will be discussed further below. Assuming there are Fₐ RTL bins from the self-attention selection 314 where Fₐ is a design parameter that we will discuss further below. Accordingly, the shape of the input Ø_{b|h}^{j}(t,f) now is(W, N, Fₐ, 2). The final preparation step is to merge the antenna and RTL dimensions, as it may result in better validation performance. Therefore, the final input dimension fed to the encoder-decoder model 316 is (W, N×Fₐ, 2).

The encoder-decoder model 316 comprises an encoder 502 that takes an input sequence and creates a dense representation of it, referred to as embedding. The embedding conveys the essence of the input to a decoder 504, which then forms a corresponding output sequence. The artificial neural network 300 uses a convolutional encoder decoder architecture where both the encoder 502 and the decoder 504 are implemented using CNNs, as shown in FIG. 5. The convolutional encoder 502 shown in FIG. 5 captures progressively high level features as the receptive fields of the network increase with the depth of the encoder 502. At each step, the encoder 502 progressively reduces the spatial resolution of the CNN feature maps through average pooling, which performs a downsampling operation. In contrast, the decoder 504 progressively increases the spatial resolution of the feature maps through up-sampling. At each layer of the encoder 502 and the decoder 504, the artificial neural network 300 uses residual connections that provide alternative paths for the gradient to flow, and allow the encoder-decoder model 316 to converge faster.

Returning to FIG. 3, loss between the target and predicted PPG signals is computed using an [symbol] 1 loss function 318. We can represent the [symbol] 1 loss as |p_{h|b}(t)−m_{|h|b}(t)|, where m_{|h|b}(t) is the predicted model output of dimension (W, 2), and p_{h|b}(t) is the ground truth PPG target of the same dimension. However, there are several challenges with the use of this loss. The first challenge is that although the artificial neural network 300 takes care in data collection to synchronize the radar and PPG sequences, there are nevertheless small synchronization errors that still remain. In experiments, we observed that the two sequences can be of with respect to each other by as much as 300 ms. With such offsets, an encoder-decoder model 316 with a [symbol] 1 loss will fail to converge. To address this issue, the artificial neural network 300 uses a sliding loss that slides the target PPG sequence p_{h|b}(t) by offsets ranging from −S to +S, computes the [symbol] 1 loss on each slide, and then selects the smallest loss value while discarding the rest. With this modification, we represent the loss L as follows:

[symbol]

=min(|p_{h|b}(t+s)−m_{h|b}(t)|) ∀−S<s<S

where S is the maximum offset amount, that is set to 300 ms in one implementation.

The second challenge is that while the PPG signal is flip-invariant, the radar phase profile is not. To understand this property, consider a case where the person 104 is facing the radar 108, and then turns around to face the opposite direction to the radar 108. As the radar signal phase captures the breathing and heart movements with respect to the radar 108, these phases will flip around as the person 104 turns around to face the other direction. However, the position of the person does not impact the PPG signal in any way. To address this challenge, the loss function is modified such that it carries this flip-invariance property. In particular, the artificial neural network 300 calculates loss on both the original and flipped target signals, and then selects the loss with the smaller value as shown by the equation:

[symbol]

=min(min(|p_{h|b}(t+s)−m_{h|b}(t)|), min(|−p_{h|b}(t+s)−m_{h|b}(t)|)) ∀−S<s<S

The third challenge is to derive first and second order derivatives from the PPG signal as they can be used to extract many informative features. However, a [symbol] 1 loss does not strictly penalize errors in the predicted first and second order derivatives of the PPG signal. Therefore, we modify the loss function 318 is modified to include terms that directly penalize both the first and second order derivatives. For simplicity, let [symbol] (x,y) represent the following:

[symbol]

(x,y)=min(min(|x(t+s)−y(t)|),min(|−x(t+s)−y(t)|))

Then, the final representation of L that includes the derivatives is as follows:

[symbol]

= [symbol] (p_{h|b},m_{h|b})+ [symbol] (p′_{h|b},m′_{h|b})+ [symbol] (p″_{h|b},m″_{h|b}) ∀−S<s<S

In one example, the encoder-decoder model 316 was trained using RMSProp optimizer for 300 epochs. A learning rate annealing routine that starts with a warm-start learning rate of 1e⁻⁴ for the first 5 epochs, 1e⁻³ for the next 195 epochs, and anneals to 2e⁻⁴ for the last 100 epochs. Training further used batch normalization after each convolution layer to get a stable distribution of input throughout training. For regularization, training used dropout layers with a probability of 0.2 after each layer of the encoder-decoder model 316.

Turning to the self-attention selection 314, the encoder-decoder model 316 translates the radar phase profile sequence to the corresponding PPG sequence. However, instead of using all F RTL bins, the artificial neural network 300 first downsized the number of bins to Fₐ. The motivation for this downsizing is to only select the RTL bins that contain either direct or multipath reflections from the person 104. Before discussing the architecture for selecting these RTL bins, we provide a motivation for why the multipath reflections are crucially important.

Consider two cases for a person where (i) the person's chest is facing the radar 108 antennas, and (ii) the person's chest is perpendicular to the radar 108 antennas. We show these two cases in FIG. 4(a-b), where the lines show the chest's exhale and inhale positions indicated by d, and the arrow shows the direction of chest's movement. The distances of the two reflections from exhale and inhale chest positions are denoted as d₁ and d₂, respectively, and let d_{c} denote the actual amount of chest movement. Also, recall that the phase of an RTL profile bin is given as

$$2 π \frac{d ( t )}{λ} .$$

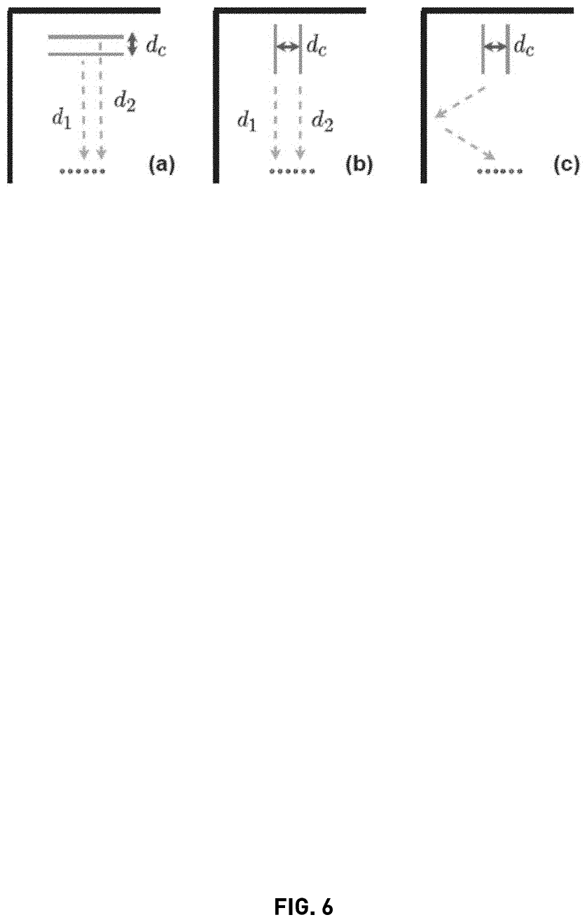

Now, in the first case in FIG. 6, the movement of the person's chest d_{c} is the same as |d₁−d₂|. Due to this change in the distance of reflection between exhale and inhale, there will be a substantial variation in phase over time due to breathing and heart movements. However, in the second case in FIG. 6, the person's chest movements do not change the magnitude of |d₁−d₂|. Hence, the direct radar reflection from the person in the second scenario will not contain any information about the person's breathing or heart movements. Now, consider a scenario in FIG. 6 where there is an additional multi path reflection that first hits the person's chest and then reflects from a nearby wall before arriving at the radar antennas. In this case, there will be a change in |d₁−d₂| and accordingly a change in the phase of signal depending on the angle of arrival of the multipath reflection. These observations show that the encoder-decoder model 316 can potentially leverage multipath reflections to substantially improve performance. Next, we discuss the self-attention selection 314 that enables the model to select the most informative RTL bins.

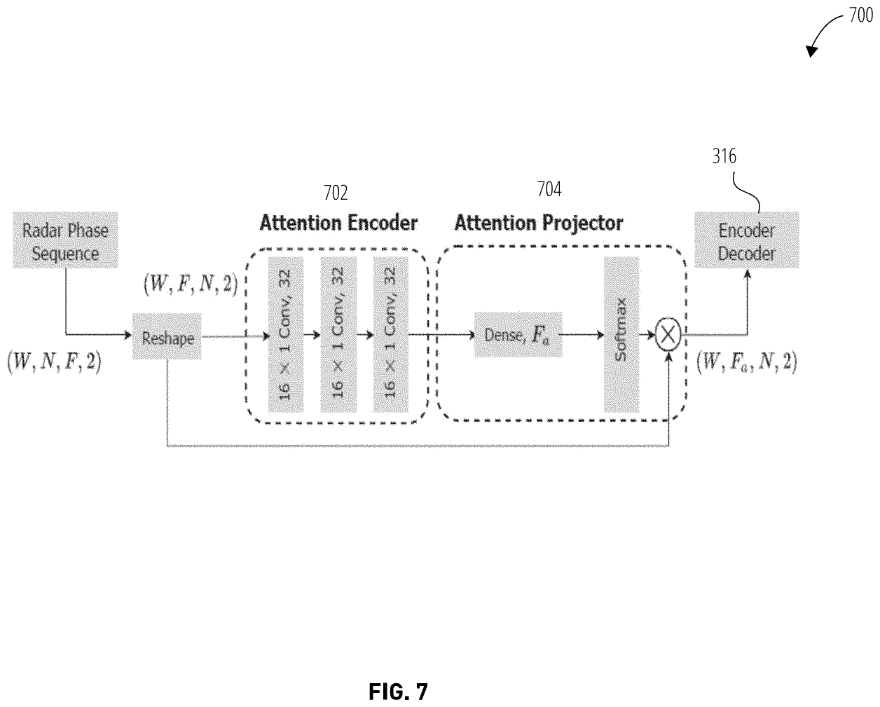

FIG. 7 is a block diagram illustrating a self-attention model architecture 700, in accordance with some examples. In one example, the self-attention selection 314 uses the self-attention model architecture 700. The self-attention model architecture 700 generates an attention map, and then projects it to the radar input to obtain a representation that contains the most informative RTL bins. An attention map is a scalar matrix that represents the relative importance of each RTL bin with respect to the target task of predicting the output PPG signal. Intuitively, we expect an RTL bin to be informative if it contains breathing and heartbeat dominant signals, and non-informative otherwise.

The self-attention model architecture 700 of an attention encoder 702 and an attention projector 704. The goal of the attention encoder 702 is to create an input representation of the input using convolution layers, whereas the goal of the attention projector 704 is to project the attention map back to the input to obtain a downsized radar phase profile representation. The encoder comprises multiple convolution layers that apply the convolution filter across the time dimension W, but keep the other input dimensions intact. Our intuition behind this choice is to independently learn features across each RTL bin. Each convolution layer is constructed similarly as in the encoder-decoder model 316.

The attention projector 704 transforms the attention encoding to a dense representation of shape (F, Fₐ), followed by a softmax layer that normalizes the output of the dense layer to produce an attention map. Let us denote the attention map with the notation Dₘₙ, where 1≤m≤F, 1≤n≤Fₐ. Recall that Fₐ denotes the number of downsized RTL bins that we want to retain after the projection step. We set Fₐ to 4 in one example implementation as it defines a rough upper limit on the number of multipaths in an indoor environment. Furthermore, we evaluated the model with different values of Fₐ), and Fₐ=4 resulted in the best performance. For the subsequent discussion, we refer to Fₐ as attention heads. An entry of the attention map Dₘₙ denotes the relative importance of the m^{th} RTL bin for the n^{th} attention head. Finally, the artificial neural network 300 multiplies the input representation with the attention map to obtain the downsized radar phase profile representation. As the self-attention model architecture 700 is a part of the artificial neural network 300, it is trained along with the encoder-decoder model 316 using the same loss function described previously.

Returning to FIG. 3, optional background removal 312 can be used to remove radar reflections related to background persons other than the person 104, if present. In this step, the artificial neural network 300 will identify the RTL bins that belong to stationary participants, i.e., the RTL bins that represent reflections from stationary participants. Recall that the shape of the radar input Ø_{b|h}^{j}(t,f) is (W, N, F, 2), where F is the number of RTL bins, set to 64 in one implementation. Before identifying the RTL bins that belong to stationary participants, the artificial neural network 300 makes two modifications to the input representation for this identification step. First, the artificial neural network 300 only considers the breathing waveform as it has a higher SNR compared to the heartbeat waveform. This is because the chest movements during breathing are of a significantly higher magnitude compared to the heart movements during the cardiac cycle. Second, the artificial neural network 300 pools the antenna dimension by summing up signals from all N antennas, as each antenna has independent measurements, and adding those measurements improves the SNR. Therefore, the modified input representation Ø_{b}(t,f) now has a shape of (W, F).

To identify the RTL bins that belong to stationary participants, when a participant is stationary, then the dominant motion is caused by the breathing activity. Therefore, taking a Fourier Transform of the phase variation of an RTL bin belonging to a stationary participant over a certain time window, then the dominant harmonic of the FFT should be in the breathing frequency range, i.e., 0.2-0.6 Hz. To implement this insight, the artificial neural network 300 uses a heuristic from that (i) checks that the highest peak of this FFT is in the breathing frequency range, and (ii) verifies that the ratio of the first and second highest peaks of the FFT is greater than a periodicity threshold η. The objective of the latter check is to verify that there are no other dominant movements such as limb or arm movements. In one example, η=2 provides a good trade-off between high true negatives (filtering RTL bins that do not belong to stationary participants) and low false negatives (filtering the RTL bins that actually reflect from stationary participants). After implementing this heuristic on each RTL bin in Ø_{b}(t,f), the artificial neural network 300 identifies \tilde{F} RTL bins that satisfy the heuristic checks.

To score the similarity of each RTL bin in \tilde{F} with a representative RTL bin F′, and then mark each bin as either a foreground or background RTL bin, the artificial neural network 300 selects the smallest bin in \tilde{F} as the representative RTL bin, which we denote as F′. This is because we define the primary participant as the one that is the closest to the device. Before scoring the comparisons, the artificial neural network 300 normalizes the input Ø_{b}(t,f) on the scale [−1, 1], where f∈\tilde{F}. Now, to compare each RTL bin with F′, the artificial neural network 300 uses Dynamic Time Warping (DTW), which is used to measure the similarity between two temporal sequences. DTW accounts for the potential differences in frequencies between the two RTL sequences. Then, the artificial neural network 300 marks the RTL bins with similarity scores greater than a similarity threshold W as the background RTL bins. Finally, the artificial neural network 300 filters out the background RTL bins so that they do not adversely affect the encoder-decoder model 316. To filter these background bins is to remove them from the radar input representation. However, this is not possible as the encoder-decoder model 316 expects inputs of fixed sizes. Instead, the artificial neural network 300 sets all the background RTL bins to zero in the original radar input representation Ø_{b|h}^{j}(t,f). After filtering the background RTL bins, the artificial neural network 300 feeds the radar input to the encoder-decoder model 316 to generate the PPG output.

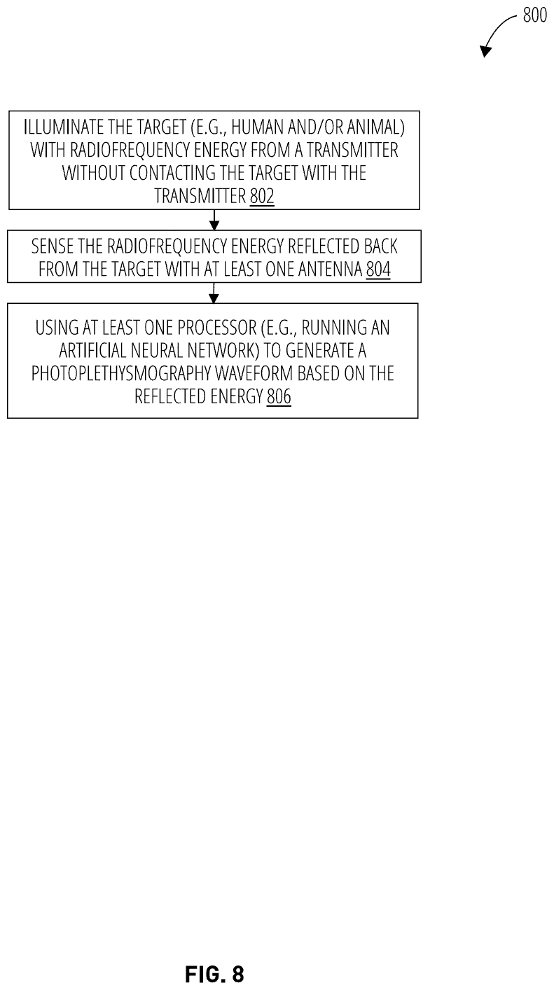

FIG. 8 illustrates a method for monitoring photoplethysmography in a target, according to some examples. In block 802, method 800 illuminates the target (e.g., human and/or animal, such as a pet) with radiofrequency energy from a transmitter without contacting the target with the transmitter. In block 804, method 800 senses the radiofrequency energy reflected back from the target with at least one antenna. In block 806, method 800 uses at least one processor (e.g., running an artificial neural network) to generate a photoplethysmography waveform based on the reflected energy.

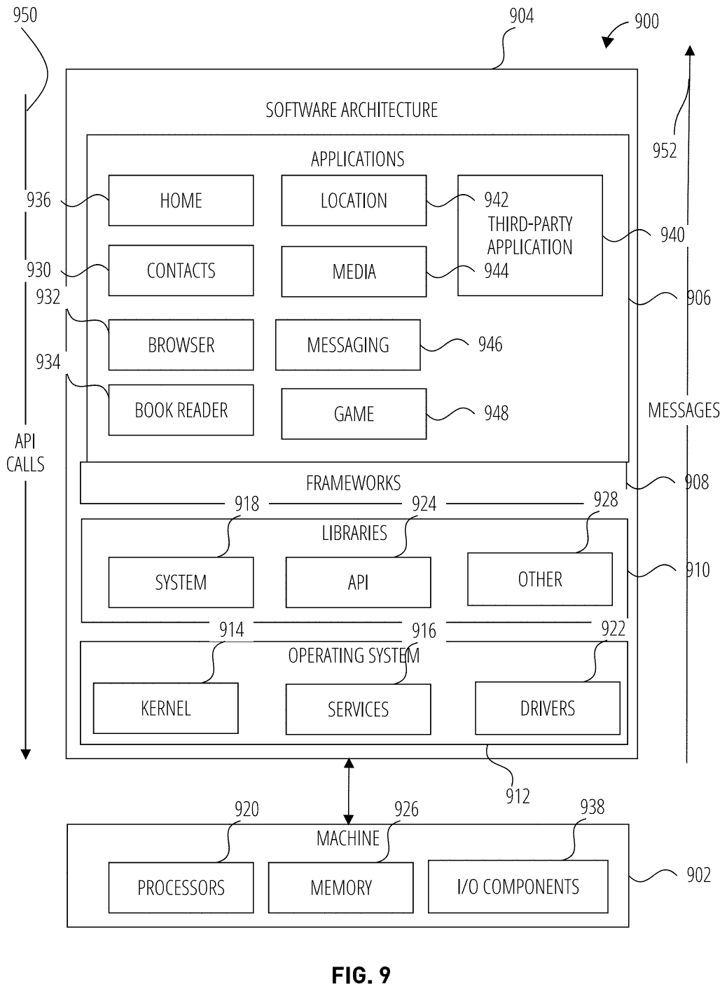

FIG. 9 is a block diagram 900 illustrating a software architecture 904, which can be installed on any one or more of the devices described herein. The software architecture 904 is supported by hardware such as a machine 902 that includes processors 920, memory 926, and I/O components 938. In this example, the software architecture 904 can be conceptualized as a stack of layers, where each layer provides a particular functionality. The software architecture 904 includes layers such as an operating system 912, libraries 910, frameworks 908, and applications 906. Operationally, the applications 906 invoke API calls 950 through the software stack and receive messages 952 in response to the API calls 950.

The operating system 912 manages hardware resources and provides common services. The operating system 912 includes, for example, a kernel 914, services 916, and drivers 922. The kernel 914 acts as an abstraction layer between the hardware and the other software layers. For example, the kernel 914 provides memory management, Processor management (e.g., scheduling), component management, networking, and security settings, among other functionalities. The services 916 can provide other common services for the other software layers. The drivers 922 are responsible for controlling or interfacing with the underlying hardware. For instance, the drivers 922 can include display drivers, camera drivers, BLUETOOTH® or BLUETOOTH® Low Energy drivers, flash memory drivers, serial communication drivers (e.g., Universal Serial Bus (USB) drivers), WI-FI® drivers, audio drivers, and power management drivers.

The libraries 910 provide a low-level common infrastructure used by the applications 906. The libraries 910 can include system libraries 918 (e.g., C standard library) that provide functions such as memory allocation functions, string manipulation functions, mathematic functions, and the like. In addition, the libraries 910 can include API libraries 924 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., Web Kit to provide web browsing functionality), and the like. The libraries 910 can also include a wide variety of other libraries 928 to provide many other APIs to the applications 906.

The frameworks 908 provide a high-level common infrastructure used by the applications 906. For example, the frameworks 908 provide various graphical user interface (GUI) functions, high-level resource management, and high-level location services. The frameworks 908 can provide a broad spectrum of other APIs that can be used by the applications 906, some of which may be specific to a particular operating system or platform.

In some examples, the applications 906 may include a home application 936, a contacts application 930, a browser application 932, a book reader application 934, a location application 942, a media application 944, a messaging application 946, a game application 948, and a broad assortment of other applications such as a third-party application 940. The applications 906 are programs that execute functions defined in the programs. Various programming languages can be employed to create one or more of the applications 906, structured in a variety of manners, such as object-oriented programming languages (e.g., Objective-C, Java, or C++) or procedural programming languages (e.g., C or assembly language). In a specific example, the third-party application 940 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 940 can invoke the API calls 950 provided by the operating system 912 to facilitate functionality described herein.

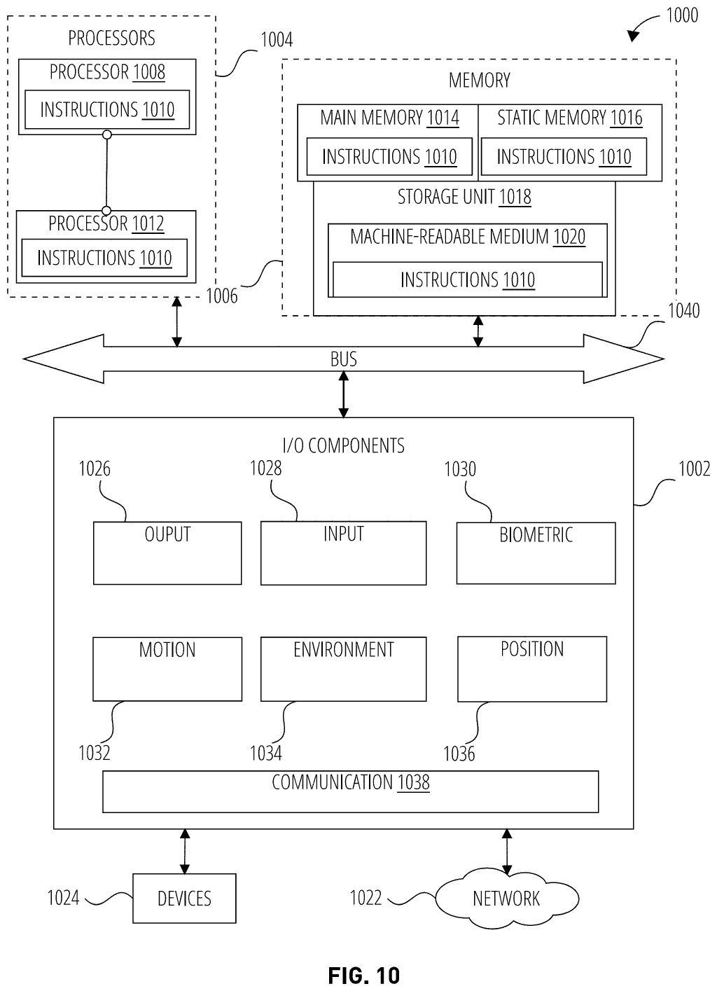

FIG. 10 is a diagrammatic representation of the machine 1000 within which instructions 1010 (e.g., software, a program, an application, an applet, an app, or other executable code) for causing the machine 1000 to perform any one or more of the methodologies discussed herein may be executed. For example, the instructions 1010 may cause the machine 1000 to execute any one or more of the methods described herein. The instructions 1010 transform the general, non-programmed machine 1000 into a particular machine 1000 programmed to carry out the described and illustrated functions in the manner described. The machine 1000 may operate as a standalone device or be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 1010, sequentially or otherwise, that specify actions to be taken by the machine 1000. Further, while a single machine 1000 is illustrated, the term “machine” may include a collection of machines that individually or jointly execute the instructions 1010 to perform any one or more of the methodologies discussed herein.

The machine 1000 may include processors 1004, memory 1006, and I/O components 1002, which may be configured to communicate via a bus 1040. In some examples, the processors 1004 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application-Specific Integrated Circuit (ASIC), a Radio-Frequency Integrated Circuit (RFIC), another Processor, or any suitable combination thereof) may include, for example, a Processor 1008 and a Processor 1012 that execute the instructions 1010. The term “Processor” is intended to include multi-core processors that may comprise two or more independent processors (sometimes referred to as “cores”) that may execute instructions contemporaneously. Although FIG. 10 shows multiple processors 1004, the machine 1000 may include a single processor with a single core, a single processor with multiple cores (e.g., a multi-core processor), multiple processors with a single core, multiple processors with multiples cores, or any combination thereof.

The memory 1006 includes a main memory 1014, a static memory 1016, and a storage unit 1018, both accessible to the processors 1004 via the bus 1040. The main memory 1006, the static memory 1016, and storage unit 1018 store the instructions 1010 embodying any one or more of the methodologies or functions described herein. The instructions 1010 may also reside, wholly or partially, within the main memory 1014, within the static memory 1016, within machine-readable medium 1020 within the storage unit 1018, within the processors 1004 (e.g., within the processor's cache memory), or any suitable combination thereof, during execution thereof by the machine 1000.

The I/O components 1002 may include various components to receive input, provide output, produce output, transmit information, exchange information, or capture measurements. The specific I/O components 1002 included in a particular machine depend on the type of machine. For example, portable machines such as mobile phones may include a touch input device or other such input mechanisms, while a headless server machine will likely not include such a touch input device. The I/O components 1002 may include many other components not shown in FIG. 10. In various examples, the I/O components 1002 may include output components 1026 and input components 1028. The output components 1026 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), or other signal generators. The input components 1028 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further examples, the I/O components 1002 may include biometric components 1030, motion components 1032, environmental components 1034, or position components 1036, among a wide array of other components. For example, the biometric components 1030 include components to detect expressions (e.g., hand expressions, facial expressions, vocal expressions, body gestures, or eye-tracking), measure biosignals (e.g., blood pressure, heart rate, body temperature, perspiration, or brain waves), or identify a person (e.g., voice identification, retinal identification, facial identification, fingerprint identification, or electroencephalogram-based identification). The motion components 1032 include acceleration sensor components (e.g., accelerometer), gravitation sensor components, rotation sensor components (e.g., gyroscope). The environmental components 1034 include, for example, one or cameras, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1036 include location sensor components (e.g., a Global Positioning System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1002 further include communication components 1038 operable to couple the machine 1000 to a network 1022 or devices 1024 via respective coupling or connections. For example, the communication components 1038 may include a network interface Component or another suitable device to interface with the network 1022. In further examples, the communication components 1038 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1024 may be another machine or any of a wide variety of peripheral devices (e.g., a peripheral device coupled via a USB).

Moreover, the communication components 1038 may detect identifiers or include components operable to detect identifiers. For example, the communication components 1038 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Data glyph, Maxi Code, PDF417, Ultra Code, UCC RSS-2D bar code, and other optical codes), or acoustic detection components (e.g., microphones to identify tagged audio signals). In addition, a variety of information may be derived via the communication components 1038, such as location via Internet Protocol (IP) geolocation, location via Wi-Fi® signal triangulation, or location via detecting an NFC beacon signal that may indicate a particular location.

The various memories (e.g., main memory 1014, static memory 1016, and/or memory of the processors 1004) and/or storage unit 1018 may store one or more sets of instructions and data structures (e.g., software) embodying or used by any one or more of the methodologies or functions described herein. These instructions (e.g., the instructions 1010), when executed by processors 1004, cause various operations to implement the disclosed examples.

The instructions 1010 may be transmitted or received over the network 1022, using a transmission medium, via a network interface device (e.g., a network interface component included in the communication components 1038) and using any one of several well-known transfer protocols (e.g., hypertext transfer protocol (HTTP)). Similarly, the instructions 1010 may be transmitted or received using a transmission medium via a coupling (e.g., a peer-to-peer coupling) to the devices 1024.

### EXAMPLES

In view of the disclosure above, various examples are set forth below. It should be noted that one or more features of an example, taken in isolation or combination, should be considered within the disclosure of this application.

1. A contactless method for monitoring photoplethysmography in a target, such as a human and/or animal (e.g., pet), the method comprising:
illuminating the target with radiofrequency energy from a transmitter without contacting the target with the transmitter;
sensing the radiofrequency energy reflected back from the target with at least one antenna; and
using at least one processor (e.g, running an artificial neural network) to generate a photoplethysmography waveform based on the reflected energy.

2. The method of example 1, wherein the processor includes a convolutional encoder-decoder model.

3. The method of any of the preceding examples, further comprising training the model using reflected radiofrequency data and photoplethysmography sensor data collected substantially simultaneously from one or more targets.

4. The method of any of the preceding examples, wherein the training further comprises estimating a round trip length of the illuminating energy to generated round trip length profiles; obtaining phase profiles of the estimated round trip length profiles over time windows; and applying bandpass filtering to the obtained phase profiles and the collected photoplethysmography sensor data.

5. The method of any of the preceding examples, further comprising resampling the reflected radiofrequency data and photoplethysmography sensor data at a common frequency before the training.

6. The method of any of the preceding examples, further comprising estimating round trip length profiles for the reflected energy, generating phase profiles from the estimated round trip lengths, and bandpass filtering the phases profiles.

7. The method of any of the preceding examples, further comprising self-attention selecting, using an attention encoder and an attention projector, the phase profiles.

8. The method of any of the preceding examples, wherein the self-attention selecting selects a radar phase profile having a multi-path reflection over a direct reflection.

9. The method of any of the preceding examples, further comprising discarding background reflections not reflected from the target.

10. The method of any of the preceding examples, further comprising applying a loss function to the sensed reflected radiofrequency energy to compensate for the target flipping.

11. A non-contact photoplethysmography detection apparatus, comprising:
a radiofrequency transmitter configured to illuminate a target, such as a human and/or animal (e.g., pet)with radiofrequency energy without contacting the target with the transmitter;
at least one antenna configured to sense the radiofrequency energy reflected back from the target; and
at least one processor (e.g., running an artificial neural network) configured to generate a photoplethysmography waveform based on the reflected energy.

12. The apparatus of example 11, wherein the processor includes a convolutional encoder-decoder model.

13. The apparatus of any of the preceding examples, wherein the convolutional encoder-decoder model is trained using reflected radiofrequency data and photoplethysmography sensor data collected substantially simultaneously from one or more targets.

14. The apparatus of any of the preceding examples, wherein the training further comprises estimating a round trip length of the illuminating energy to generate round trip length profiles; obtaining phase profiles of the estimated round trip length profiles over time windows; and applying bandpass filtering to the obtained phase profiles and the collected photoplethysmography sensor data.

15. The apparatus of any of the preceding examples, wherein the at least one processor is further configured to resample the reflected radiofrequency data and photoplethysmography sensor data at a common frequency before the training.

16. The apparatus of any of the preceding examples, wherein the at least one processor is further configured to estimate round trip length profiles for the reflected energy, generate phase profiles from the estimated round trip lengths, and bandpass filter the phases profiles.

17. The apparatus of any of the preceding examples, wherein the at least one processor is further configured to self-attention select, using an attention encoder and an attention projector, the phase profiles.

18. The apparatus of any of the preceding examples, wherein the self-attention selecting selects a radar phase profile having a multi-path reflection over a direct reflection.

19. The apparatus of any of the preceding examples, wherein the at least one processor is further configured to discard background reflections not reflected from the target.

20. The apparatus of any of the preceding examples, wherein the at least one processor is further configured to apply a loss function to the sensed reflected radiofrequency energy to compensate for the target flipping.

21. A non-contact photoplethysmography detection apparatus comprising:
at least one processor; and
a non-transitory memory having stored thereon instructions to cause the at least one processor execute the method of any of examples 1-10.

22. A non-transitory computer-readable memory having stored thereon instructions to cause the computer to execute the method of any of the examples 1-10.

### Glossary

“Carrier Signal” refers to any intangible medium capable of storing, encoding, or carrying instructions for execution by the machine, and includes digital or analog communications signals or other intangible media to facilitate communication of such instructions. Instructions may be transmitted or received over a network using a transmission medium via a network interface device.

“Communication Network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network, and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth-generation wireless (4G) networks, Universal Mobile Telecommunications System (UMTS), High-Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner In examples, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC).A hardware component may also include programmable logic or circuitry that is temporarily configured by software to perform certain operations. For example, a hardware component may include software executed by a general -purpose processor or other programmable processor. Once configured by such software, hardware components become specific machines (or specific components of a machine) uniquely tailored to perform the configured functions and are no longer general-purpose processors. A decision to implement a hardware component mechanically, in dedicated and permanently configured circuitry, or in temporarily configured circuitry (e.g., configured by software), may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering examples in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In examples in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of methods described herein may be performed by one or more processors 1004 or processor-implemented components. Moreover, the one or more processors may also operate to support performance of the relevant operations in a “cloud computing” environment or as a “software as a service” (SaaS).For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some examples, the processors or processor-implemented components may be located in a single geographic location (e.g., within a home environment, an office environment, or a server farm). In some examples, the processors or processor-implemented components may be distributed across a number of geographic locations.

“Computer-Readable Medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine-Storage Medium” refers to a single or multiple storage devices and/or media (e.g., a centralized or distributed database, and/or associated caches and servers) that store executable instructions, routines and/or data. The term includes solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium”, “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Module” refers to logic having boundaries defined by function or subroutine calls, branch points, Application Program Interfaces (APIs), or other technologies that provide for the partitioning or modularization of particular processing or control functions. Modules are typically combined via their interfaces with other modules to carry out a machine process. A module may be a packaged functional hardware unit designed for use with other components and a part of a program that usually performs a particular function of related functions. Modules may constitute either software modules (e.g., code embodied on a machine-readable medium) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein. In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a Field-Programmable Gate Array (FPGA) or an Application Specific Integrated Circuit (ASIC). A hardware module may also include programmable logic or circuitry that is temporarily configured by software to perform certain operations. For example, a hardware module may include software executed by a general-purpose processor or other programmable processor. Once configured by such software, hardware modules become specific machines (or specific components of a machine) uniquely tailored to perform the configured functions and are no longer general-purpose processors. It will be appreciated that the decision to implement a hardware module mechanically, in dedicated and permanently configured circuitry, or in temporarily configured circuitry (e.g., configured by software) may be driven by cost and time considerations. Accordingly, the phrase “hardware module”(or “hardware-implemented module”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where a hardware module comprises a general-purpose processor configured by software to become a special-purpose processor, the general-purpose processor may be configured as respectively different special-purpose processors (e.g., comprising different hardware modules) at different times. Software accordingly configures a particular processor or processors, for example, to constitute a particular hardware module at one instance of time and to constitute a different hardware module at a different instance of time. Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information). The various operations of example methods and routines described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors. Similarly, the methods described herein may be at least partially processor-implemented, with a particular processor or processors being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)). The performance of certain of the operations may be distributed among the processors, not only residing within a single machine, but deployed across a number of machines. In some example embodiments, the processors or processor-implemented modules may be located in a single geographic location (e.g., within a home environment, an office environment, or a server farm). In other example embodiments, the processors or processor-implemented modules may be distributed across a number of geographic locations.

“Processor” refers to any circuit or virtual circuit (a physical circuit emulated by logic executing on an actual processor) that manipulates data values according to control signals (e.g., “commands”, “op codes”, “machine code”, etc.) and which produces corresponding output signals that are applied to operate a machine. A processor may, for example, be a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC) or any combination thereof. A processor may further be a multi-core processor having two or more independent processors (sometimes referred to as “cores”) that may execute instructions contemporaneously.

“Signal Medium” refers to any intangible medium that is capable of storing, encoding, or carrying the instructions for execution by a machine and includes digital or analog communications signals or other intangible media to facilitate communication of software or data. The term “signal medium” may o include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

What is claimed is:

1. A contactless method for monitoring photoplethysmography in a target, the method comprising:
illuminating the target with radiofrequency energy from a transmitter without contacting the target with the transmitter;
sensing the radiofrequency energy reflected back from the human with at least one antenna; and
using at least one processor to generate a photoplethysmography waveform based on the reflected energy.

2. The method of claim 1, wherein the at least one processor includes a convolutional encoder-decoder model.

3. The method of claim 2, further comprising training the model using reflected radiofrequency data and photoplethysmography sensor data collected substantially simultaneously from one or more targets.

4. The method of claim 3, wherein the training further comprises estimating a round trip length of the illuminating energy to generated round trip length profiles; obtaining phase profiles of the estimated round trip length profiles over time windows; and applying bandpass filtering to the obtained phase profiles and the collected photoplethysmography sensor data.

5. The method of claim 3, further comprising resampling the reflected radiofrequency data and photoplethysmography sensor data at a common frequency before the training.

6. The method of claim 1, further comprising estimating round trip length profiles for the reflected energy, generating phase profiles from the estimated round trip lengths, and bandpass filtering the phases profiles.

7. The method of claim 6, further comprising self-attention selecting, using an attention encoder and an attention projector, the phase profiles.

8. The method of claim 7, wherein the self-attention selecting selects a radar phase profile having a multi-path reflection over a direct reflection.

9. The method of claim 1, further comprising discarding background reflections not reflected from the target.

10. The method of claim 1, further comprising applying a loss function to the sensed reflected radiofrequency energy to compensate for the target flipping.

11. A non-contact photoplethysmography detection apparatus, comprising:
a radiofrequency transmitter configured to illuminate a target with radiofrequency energy without contacting the target with the transmitter;
at least one antenna configured to sense the radiofrequency energy reflected back from the target; and
at least one processor configured to generate a photoplethysmography waveform based on the reflected energy.

12. The apparatus of claim 11, wherein the at least one processor includes a convolutional encoder-decoder model.

13. The apparatus of claim 12, wherein the convolutional encoder-decoder model is trained using reflected radiofrequency data and photoplethysmography sensor data collected substantially simultaneously from one or more targets.

14. The apparatus of claim 13, wherein the training further comprises estimating a round trip length of the illuminating energy to generate round trip length profiles; obtaining phase profiles of the estimated round trip length profiles over time windows; and applying bandpass filtering to the obtained phase profiles and the collected photoplethysmography sensor data.

15. The apparatus of claim 13, wherein the at least on processor is further configured to resample the reflected radiofrequency data and photoplethysmography sensor data at a common frequency before the training.

16. The apparatus of claim 11, wherein the at least one processor is further configured to estimate round trip length profiles for the reflected energy, generate phase profiles from the estimated round trip lengths, and bandpass filter the phases profiles.

17. The apparatus of claim 16, wherein the at least one processor is further configured to self-attention select, using an attention encoder and an attention projector, the phase profiles.

18. The apparatus of claim 17, wherein the self-attention selecting selects a radar phase profile having a multi-path reflection over a direct reflection.

19. The apparatus of claim 11, wherein the at least one processor is further configured to discard background reflections not reflected from the target.

20. The apparatus of claim 11, wherein the at least one processor is further configured to apply a loss function to the sensed reflected radiofrequency energy to compensate for the target flipping.
